SEQUENCE LISTING (4 sequences)

SEQ ID NO: 1 - DNA, 19 bp, synthetic construct
  source 1..19: mol_type = other DNA
caggcggtgcctatgtctc

SEQ ID NO: 2 - DNA, 22 bp, synthetic construct
  source 1..22: mol_type = other DNA
cgatcaccccgaagttcagtag

SEQ ID NO: 3 - DNA, 24 bp, synthetic construct
  source 1..24: mol_type = other DNA
tcaacagcaactcccactcttcca

SEQ ID NO: 4 - DNA, 24 bp, synthetic construct
  source 1..24: mol_type = other DNA
accctgttgctgtagccgtattca

(19) United States
(12) Patent Application Publication
(10) Pub. No.: US 2024/0269104 A1
(43) Pub. Date: Aug. 15, 2024

(54) METHOD FOR TREATING PARKINSON'S DISEASE

(71) Applicant: Olatec Therapeutics, Inc., New York, NY (US)

(72) Inventors: Charles A. Dinarello, Boulder, CO (US); Jesus Amo Aparicio, Denver, CO (US)

(21) Appl. No.: 18/444,049

(22) Filed: Feb. 16, 2024

Related U.S. Application Data
(63) Continuation of application No. PCT/US2022/075219, filed on Aug. 19, 2022.
(60) Provisional application No. 63/235,064, filed on Aug. 19, 2021.

(51) Int. Cl. A61K 31/275; A61P 25/16

(52) U.S. Cl. CPC A61K 31/275; A61P 25/16

(57) ABSTRACT

The present invention is directed to a method for treating Parkinson's disease. The method comprises administering to a subject in need thereof dapansutrile, in an effective amount. Dapansutrile minimizes the clinical features of PD such as locomotor impairments through the modulation of the inflammatory response, reduction in α-synuclein levels, and protection of dopaminergic neurons. A preferred route of administration is oral administration.

### CROSS-REFERENCE TO RELATED APPLICATION

This application is a continuation of PCT/US2022/075219, filed Aug. 19, 2022; which claims the benefit of U.S. Provisional Application No. 63/235,064, filed Aug. 19, 2021. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on Aug. 19, 2022 is named Sequence Listing.xml and is 4500 bytes in size.

### TECHNICAL FIELD

The present invention is directed to a method for treating Parkinson's disease by administering an effective amount of dapansutrile.

### BACKGROUND

Parkinson's disease (PD), also known as idiopathic or primary parkinsonism, is a degenerative neurological disorder of the central nervous system. PD is the second most frequent degenerative disorder after Alzheimer's disease. The motor symptoms of PD result from the death of dopamine-generating cells in the substantia nigra, a region of the midbrain; the cause of this cell death is unknown. Early in the course of the disease, the most obvious symptoms are movement-related; these include shaking, rigidity, slowness of movement and difficulty with fine motor skills, walking, and gait. Later, thinking and behavioral problems may arise, with dementia commonly occurring in the advanced stages of the disease, whereas depression is the most common psychiatric symptom. Other symptoms include sensory, sleep and emotional problems.

Clinically, its cardinal features include tremor, muscle rigidity, slowness of voluntary movement and postural instability. Although PD neuropathology encompasses a number of different neurotransmitter pathways, the disabling manifestations cited above are attributed primarily to a deficit of dopamine in the brain due to the death of dopaminergic neurons.

The pathological hallmark of PD is a loss of dopaminergic neurons in the substantia nigra pars compacta resulting in a reduction in the levels of the neurotransmitter dopamine (DA) at the striatum. Since DA plays a role in the modulation of some brain circuits related with movement, its reduction leads to motor impairments. Although therapeutics approved for PD are available, most focus on the reestablishment of DA levels. Nevertheless, with current therapeutics that increases DA levels in the brain, the deficits persist, and treatments are unable to effectively slow or halt disease progression.

There is a need for a method for treating PD to prevent the progression of this disease. The method should be effective and have no significant side effects.

### BRIEF DESCRIPTION OF THE DRAWING

FIG. 1 shows therapeutic approach of using dapansutrile (OLT1177®) to treat PD.

FIG. 2 shows how dapansutrile (OLT1177®) works to treat an experimental PD model (MPTP model).

FIG. 3 shows the effect of dapansutrile on the rotarod performances at day 3 after MPTP injections. Mice were treated with dapansutrile through oral gavage 30 minutes before the MPTP injections. The overall rod performance (ORP) of each animal was then calculated as the area under the curve in a plot of time-on-the-rod against rotation speed. N=4 for saline. N=9 for MPTP+saline. N=7 for MPTP+dapansutrile. One-way ANOVA with Dunnett's post hoc correction. ** p<0.001.

FIG. 4 shows TNF RNA fold change in the brain 1 day after MPTP injection. Mice were treated with dapansutrile through oral gavage 30 minutes before the MPTP injections. N=5 for MPTP+saline. N=5 for MPTP+dapansutrile. ** p<0.01. T-test comparisons with Holm-Sidak's post hoc correction.

FIG. 5 shows protein levels of IL-18 in the brain at 3 days after MPTP injection. Mice were treated with dapansutrile through oral gavage 30 minutes before the MPTP injections. N=4 for MPTP+saline. N=3 for MPTP+dapansutrile. * p<0.05. T-test comparisons with Holm-Sidak's post hoc correction.

FIG. 6 shows the effect of anakinra on the rotarod performances at day 3 after injections. Mice were treated with anakinra through intraperitoneal injection 30 minutes before MPTP. N=4 for saline. N=5 for MPTP+saline. N=5 for MPTP+anakinra. One-way ANOVA with Dunnett's post hoc correction. * p<0.05.

FIG. 7 shows brain levels of dapansutrile in the absence of an exogenous stimulus. Mice were fed with dapansutrile-fortified pellets (OLT, 7.5 g per kg of pellet) for three weeks. Pellets with the same composition except dapansutrile were used as standard diet. Data are represented as mean±SEM. N=5 per group. T-test comparison was used to analyze differences between groups. *** p<0.001.

FIGS. 8A-8B show dapansutrile improves the motor performance of mice three days after MPTP-acute administration. FIG. 8A shows time to fall on the accelerating rotarod device. Mice received dapansutrile either at 60 mg/kg (OLT 60) or 200 mg/kg (OLT 200) once each day starting one hour before MPTP administration. Saline was used as the vehicle. Sham mice that did not receive MPTP were used as control. FIG. 8B shows running pattern of the mice at the different speeds on the accelerating rotarod device. Data are pooled from three separate experiments and represented as mean±SEM for FIG. 8A and mean for FIG. 8B. N=9 for sham+saline, N=12 for MPTP+saline, N=9 for MPTP+OLT 60, and N=14 for MPTP+OLT 200. One-way ANOVA with Tukey's post hoc correction was used to analyze differences between groups for FIG. 8A with * p<0.05, ** p<0.01, and *** p<0.001. Kaplan-Meier test was used to analyze differences between groups for FIG. 8B with **** p<0.0001 for MPTP+OLT 200 against MPTP+saline.

FIG. 9 shows number of different cell types in the blood after treatment with 200 mg/kg of dapansutrile (OLT) in MPTP-injected mice. White blood cells (WBC), lymphocytes (LYM), monocytes (MONO), and granulocytes (GRAN) were measured from the peripheral blood at day seven after MPTP administration. Data are represented as mean±SEM. N=5 for sham+saline, N=6 for MPTP+saline, and N=6 for MPTP+OLT. Two-way ANOVA with Tukey's post hoc correction was used to analyze differences between groups. * p<0.05 and **p<0.01.

FIGS. 10A-10B show cytokine levels in splenocytes from MPTP-injected mice. Production of IL-1a (FIG. 10A) and IL-6 (FIG. 10B) after two days of culture with LPS (1 μg/ml and 5 μg/ml) as the stimulant. Medium without LPS was used as control. Levels of IL-1α were measured in the lysate cells whereas IL-6 was measured in the supernatant. Spleens were isolated and cultured at seven after MPTP administration. Mice were treated with 200 mg/kg of dapansutrile (OLT) for seven days. Saline was used as vehicle. Data are represented as mean±SEM. N=5 for sham+saline and N=6 for MPTP+saline and MPTP+OLT. One-way ANOVA with Tukey's post hoc correction was used to analyze differences between groups. * p<0.05 and ** p<0.01.

FIGS. 11A-11H show dapansutrile modulates the dynamics of pro-inflammatory cytokines in the brain at different time points after MPTP-acute administration. FIGS. 11A-11D show brain levels of IL-1β (FIGS. 11A-11B) and IL-18 (FIGS. 11C-11D) in the striatum (St) and ventral midbrain (VM) four days after MPTP administration. FIGS. 11E-11H show brain levels of IL-6 (FIGS. 11E-11F) and IL-17A (FIGS. 11G-11H) seven days after MPTP administration. Mice were treated with 200 mg/kg of dapansutrile (OLT) once each day starting one hour before MPTP administration. Saline was used as the vehicle. Data are represented as mean±SEM. N=4 per group for FIGS. 11A-11D and N=5 per groups for FIGS. 11E-11H. One-way ANOVA with Tukey's post hoc correction was used to analyze differences between groups. * p<0.05, ** p<0.01, and *** p<0.001.

FIGS. 12A-12C show dapansutrile increases the levels of TREM2 after MPTP-acute administration. FIG. 12A is representative immunoblotting showing the levels of glycosylated (35 kDa) and non-glycosylated (28 kDa) forms of TREM2 in the striatum four days after MPTP administration. Mice were treated with 200 mg/kg of dapansutrile (OLT) once each day starting one hour before MPTP administration. β-actin was used as loading control. Molecular weight (kDa) is marked on the right side. FIGS. 12B-12C are quantification of 28 kDa (FIG. 12B) and 35 kDa (FIG. 12C) forms of TREM2. N=4 per group. One-way ANOVA with Tukey's post hoc correction was used to analyze differences between groups. * p<0.05 and ** p<0.01.

FIGS. 13A-13C show dapansutrile reduces the levels of α-synuclein in the ventral midbrain after MPTP-subacute administration. FIG. 13A is representative immunoblotting showing the levels of monomeric and oligomeric forms of α-synuclein in the different groups. Mice were treated with 200 mg/kg of dapansutrile (OLT) for 6 days. Saline was used as the vehicle. β-actin was used as loading control. Molecular weight (kDa) is marked on the right side. FIGS. 13B-13C are quantification of monomeric (FIG. 13B) and oligomeric (FIG. 13C) forms of α-synuclein. N=4 for sham+saline and N=5 for MPTP+saline and MPTP+OLT. One-way ANOVA with Tukey's post hoc correction was used to analyze differences between groups. * p<0.05 and ** p<0.01.

FIGS. 14A-14D show dapansutrile protects dopaminergic neurons from MPTP-mediated cell death. FIGS. 14A-14B are representative tyrosine hydroxylase (TH) immunostaining in the striatum (St, FIG. 14A) and substantia nigra pars compacta (SNpc, FIG. 14B). Mice were treated with 200 mg/kg of dapansutrile (OLT) for 7 days. Saline was used as the vehicle. Positive signal appears in brown color. The area outlined in the box is shown in higher magnification in the inset. FIGS. 14C-14D show TH immunoreactivity in the St (FIG. 14C) and TH stereological counting in the SNpc (FIG. 14D). Data are represented as mean±SEM. N=5 for sham+saline and N=7 for MPTP groups. One-way ANOVA with Tukey's post hoc correction was used to analyze differences between groups. * p<0.05, ** p<0.01, and p<0.001.

### DETAILED DESCRIPTION

The present invention provides a method of treating PD using dapansutrile.

The inventors have discovered that dapansutrile crosses the blood-brain barrier and gets into the brain. Dapansutrile then inhibits the processing on IL-1β by inflammatory cells in the brain and reduces the expression of pro-inflammatory cytokines IL-1β, IL-18, IL-6, IL-17A in the brain.

Dapansutrile minimizes the clinical features of PD through the modulation of the inflammatory response, reduction in α-synuclein levels, and protection of dopaminergic neurons.

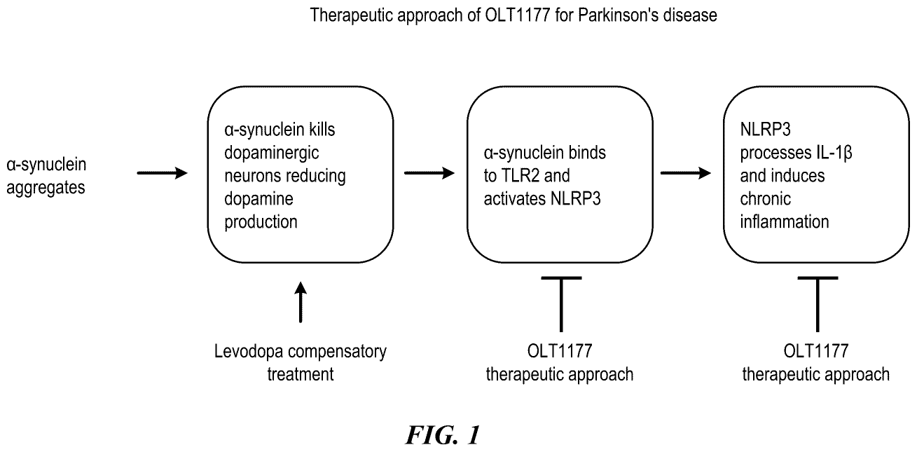

FIG. 1 shows therapeutic approach of using dapansutrile (OLT1177®) to treat PD.

### Compound

The present invention uses a purified compound of dapansutrile (3-methanesulfonylpropionitrile), or a pharmaceutically acceptable solvate thereof:

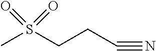

“Solvate,” as used herein, is an addition complex in which the compound is combined with an acceptable solvent in some fixed proportion. Acceptable solvents include, but are not limited to, water, acetic acid, ethanol, and other appropriate organic solvents for dapansutrile.

“Pharmaceutically acceptable solvate,” as used herein, is a solvate that retain the desired biological activity of the parent compound and do not impart undesired toxicological effects.

### Pharmaceutical Compositions

The active compound dapansutrile, or its pharmaceutically acceptable solvate in the pharmaceutical compositions in general is in an amount of about 0.1-5% for an injectable formulation, 0.1-5% for a patch formulation, about 1-90% for a tablet formulation, 1-100% for a capsule formulation, and about 0.01-20%, or 0.05-20%, or 0.1-20%, or 0.2-15%, or 0.5-10%, or 1-5% (w/w) for a topical formulation.

In one embodiment, the pharmaceutical composition is in a dosage form such as tablets, capsules, granules, fine granules, powders, syrups, suppositories, injectable solutions, patches, or the like.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cystein, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and ploxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable carriers may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of dapansutrile may contain other excipients that have no bioactivity and no reaction with the active compound. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, starch, and tragacanth gum, poly(acrylic acid), and polyvinylpyrrolidone.

For example, a patch formulation of dapansutrile may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters (e.g., lauryl lactate) or diethylene glycol monoethylether.

Topical formulations including dapansutrile can be in a form of gel, cream, lotion, liquid, emulsion, ointment, spray, solution, and suspension. The inactive ingredients in the topical formulations for example include, but not limited to, lauryl lactate (emollient/permeation enhancer), diethylene glycol monoethylether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent). In one embodiment, diethylene glycol monoethylether is included in the topical gel formulation.

### Method of Use

Liu et al (Mol Neurobiol 54, 7762-7776, 2017) report that IL-17A from Th17 cells kills dopaminergic neurons by interaction between two adhesion molecules LFA-1/ICAM-1 expressed on the membrane of these cells. Liu et al (Brain Behav Immun 81, 630-645, 2019) report that IL-17A deficiency or blockade avoids dopaminergic neurodegeneration and motor impairments in MPTP models, and IL-17A is associated with detrimental effects in a mouse model of PD.

Administration of dapansutrile reduces the levels of pro-inflammatory cytokines in the brain. Changes are observed not only in IL-1β and IL-18 as NLRP3 direct cytokines, but also in IL-6 and IL-17A as downstream cytokines, which indicate that dapansutrile is effective at modulating the adaptive immune response in PD. Dapansutrile preserves the body's immune surveillance by reducing pro-inflammatory cytokines and by protecting from cell death.

The aggregation of the intracellular α-synuclein is considered the primary pathogenic cause of dopaminergic neuron death. Reports by Wang et al (Proc. Natl. Acad. Sci. 113:9587-9592, 2016) and Bassil et al (Proc. Natl. Acad. Sci. 113:9593-9598, 2016) indicate that caspase-1-mediated truncation of α-synuclein is an upstream event culminating in aggregation and toxicity of this molecule in synucleinopathies. α-synuclein accumulation results in the death of dopaminergic neurons in the brain and in the initiation of a chronic inflammatory response that translates into secondary damage.

Dapansutrile is effective in modulating inflammatory response and reducing the aggregation of α-synuclein in the brain, which results in the protection of dopaminergic neurons and improvement in the locomotor performance.

The present invention is directed to a method of treating PD, as well as related synucleinopathies, e.g. dementia with Lewy bodies (DLB), Lewy body disease (LBD), multiple system atrophy (MSA). The method comprises the step of administering to a subject in need thereof an effective amount of dapansutrile. “An effective amount,” as used herein, is the amount effective to treat a disease by ameliorating the pathological condition or reducing the symptoms of the disease. Dapansutrile can be used as a monotherapy or as a combination therapy with other drug treatment.

The present invention provides a therapeutic intervention for neuroinflammation associated with synucleinopathies, e.g., PD, by targeting α-synuclein aggregation resulting from caspase-1 mediated α-synuclein truncation. Dapansutrile treatment also targets the maturation and release of pro-IL-1β and pro-IL-18 into inflammatory cytokines by activated microglia downstream of α-synuclein released from neurons. The blockade of the inflammatory response due to dapansutrile may result in a reduction in the activation/infiltration of immune cell subsets on the brain: microglia, macrophages, and neutrophils. Dapansutrile reduces the death of dopaminergic neurons.

The present method reduces or alleviates motor dysfunction or behavioral dysfunction in a PD patient. For example, the method improves movement-related symptoms such as shaking, rigidity, slowness of movement, difficulty with fine motor skills, walking, and gait.

The inventors have demonstrated that dapansutrile has a beneficial effect on an animal model of PD. The inventors have shown that dapansutrile reverts the functional deficits associated to MPTP injection.

The pharmaceutical composition of the present invention can be applied by systemic administration or local administration. Systemic administration includes, but is not limited to oral, parenteral (such as intravenous, intramuscular, subcutaneous or rectal), and inhaled administration. In systemic administration, the active compound first reaches plasma and then distributes into target tissues. Oral administration is a preferred route of administration for the present invention. Local administration includes topical administration.

Dosing of the composition can vary based on the extent of the injury and each patient's individual response. For systemic administration, plasma concentrations of the active compound delivered can vary; but are generally 1×10⁻¹⁰-1×10⁻⁴ moles/liter, and preferably 1×10⁻⁸-1×10⁻⁵ moles/liter.

Systemic administration includes oral, parenteral (such as intravenous, intramuscular, subcutaneous or rectal), and other systemic routes of administration. In systemic administration, the active compound first reaches plasma and then distributes into target tissues.

In one embodiment, the pharmaceutical composition is administrated orally to the subject. The dosage for oral administration is generally at least 0.1 mg/kg/day and less than 100 mg/kg/day or 200 mg/kg/day. For example, the dosage for oral administration is 1-100, or 5-50, or 10-50 mg/kg/day, for a human subject. For example, the dosage for oral administration is 100-10,000 mg/day, and preferably 500-2000, 500-4000, 500-4000, 1000-5000, 2000-5000, 2000-6000, or 2000-8000 mg/day for a human subject. The drug can be orally taken once, twice, three times, or four times a day.

In one embodiment, the pharmaceutical composition is administrated intravenously to the subject. The dosage for intravenous bolus injection or intravenous infusion is generally 0.03 to 20 and preferably 0.03 to 10 mg/kg/day.

In one embodiment, the pharmaceutical composition is administrated subcutaneously to the subject. The dosage for subcutaneous administration is generally 0.3-20, and preferably 0.3-3 mg/kg/day.

In one embodiment, the composition is applied topically. The composition is topically applied at least 1 or 2 times a day, or 3 to 4 times per day, depending on the medical issue and the disease pathology. In general, the topical composition comprises about 0.01-20%, or 0.05-20%, or 0.1-20%, or 0.2-15%, 0.5-10, or 1-5% (w/w) of the active compound. Typically, 0.2-10 mL of the topical composition is applied to the individual per dose.

Those of skill in the art will recognize that a wide variety of delivery mechanisms are also suitable for the present invention.

The present invention is useful in treating a mammal subject, such as humans, horses, dogs and cats. The present invention is particularly useful in treating humans.

The following examples further illustrate the present invention. These examples are intended merely to be illustrative of the present invention and are not to be construed as being limiting.

### EXAMPLES

### Example 1. MPTP Model

Among the various models of PD, the MPTP (1-methyl-4-phenyl-1,2,3,6-tetrahydropyridine) model has become the most commonly used. The injection of this toxin into the mouse intraperitoneal space produces a reliable and reproducible lesion of the nigrostriatal dopaminergic pathway.

MPTP (Sigma-Aldrich, St. Louis, MO) can be administered at two dose regimens: acute and sub-acute. For the acute regimen, MPTP was administered intraperitoneally (i.p.) four times in one day. Administrations were performed every two hours over an eight-hour period. For the subacute regimen, mice received a single i.p. administration of MPTP per day for five consecutive days. A 20 mg/kg free-base MPTP dose was used for every dose in both models. Sterile saline was used as the vehicle. Control mice were injected with saline solution using the same administration protocol and were defined as sham. Mice were sacrificed at different days after the first injection of MPTP. We used the acute protocol in C56BL/J mice to study dopamine neurodegeneration, inflammation, and motor deficits, whereas the subacute model was used to evaluate α-synuclein aggregation in the following examples.

### Example 2. Dapansutrile Improves the Locomotor Deficits

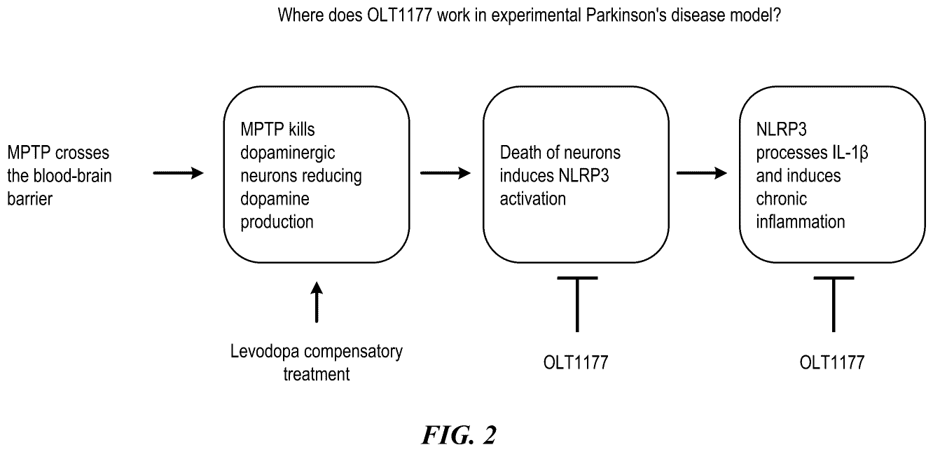

FIG. 2 shows how dapansutrile (OLT1177®) works to treat an experimental PD model (MPTP model).

We investigated overall rotarod performance (ORP) test for evaluating overall locomotory ability in the MPTP-injected-mouse model of PD. For this procedure, mice were pretrained on the rotarod and then tested at a series of increasing speeds, the time that the animal remained on the rod at each speed was recorded; the overall rod performance (ORP) of each animal was then calculated as the area under the curve in a plot of time-on-the-rod against rotation speed.

The accelerating rotarod (Ugo Basile, Gemonio, ITALY) was used to evaluate locomotor function in mice subjected to the MPTP-acute administration. An accelerating program was selected to detect locomotor impairments associated to MPTP (Keshet et al, J Comp Neurol 504:690-701, 2007; Rui et al., Int J Neuropsychopharmacol., 2020). We used a rotarod device with a cylinder rod of three centimeters in diameter. The mice underwent a training period of three consecutive days prior to MPTP administration. In each training session, mice were subjected to run at four constant speeds (10 rpm, 20 rpm, 30 rpm, and 40 rpm) for 150 seconds at each speed. If mice fell during that time, they were put back in the rotarod immediately. On the testing day, we used an increased-speed program. Mice were placed on the rotarod device for 150 seconds at 30 rpm as warm up. Immediately after 150 seconds, the accelerating session started, and the speed increased from 30 rpm to 70 rpm for 500 seconds. Time to fall was recorded. Accelerating sessions were repeated three times with a resting interval of five minutes between sessions. Average time to fall per mouse was calculated and used as final measurement.

We then evaluated the effect of dapansutrile. To avoid overloading animals with fluid (which can lead to fatal heart failure), dapansutrile was administered through oral gavage at 200 mg/kg in water. Dapansutrile was given 30 minutes before the first injection of MPTP. Mice treated with saline were used as control. We also included a sham-group that received saline through intraperitoneal injections instead of MPTP.

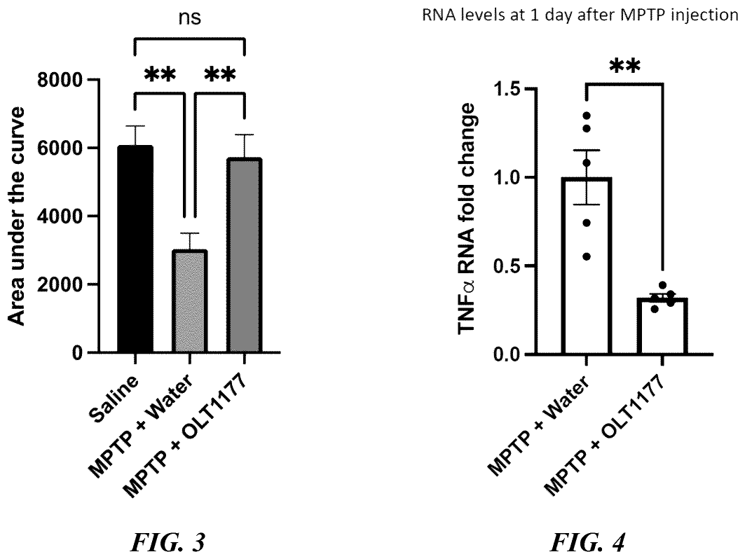

At day 3 after MPTP injection, we observed that mice treated with dapansutrile showed an increased latency to fall. Dapansutrile-treated mice stayed at the rotarod around 90 seconds more than mice treated with water. Moreover, there were not significant differences between dapansutrile-treated group and the sham group. The overall rod performance (ORP) of each animal was calculated as the area under the curve in a plot of time-on-the-rod against rotation speed. (FIG. 3).

### Example 3. Dapansutrile Reduces the TNFα RNA Levels in the Brain

Mice were treated dapansutrile or saline and injected with MPTP as described in Example 2.

24 hours after the first injection of MPTP, mice were sacrificed. A piece of brain, centered in the striatum, was taken from each mouse and frozen in liquid nitrogen. Samples were homogenized with Trizol lysis reagent (Thermo Fisher) following the manufacturer's protocol. 1 μg of RNA from each sample was reversely transcribed using the High-Capacity Reverse Transcription kit (Applied BioSystems).

Gene expression measures were performed through qPCR using SYBR Green master mix (Applied Biosystems) and recommended thermal cycling parameters: 1 cycle of polymerase activation for 10 min at 95° C. and 40 cycles of denaturalization for 15 sec at 95° C. plus annealing/extension for 1 min at 60° ° C. (capture). Gene levels of TNFα (a pro-inflammatory cytokine) were normalized to the relation ratio of expression of the GADPH gene following the ΔΔCT method. Primers sequences are detailed below. Final fold-change values were obtained by normalization with naïve condition.

TNF-F:
(SEQ ID NO: 1)
CAGGCGGTGCCTATGTCTC
TNF-R:
(SEQ ID NO: 2)
CGATCACCCCGAAGTTCAGTAG
GAPDH-F:
(SEQ ID NO: 3)
TCAACAGCAACTCCCACTCTTCCA
GAPDH-R:
(SEQ ID NO: 4)
ACCCTGTTGCTGTAGCCGTATTCA

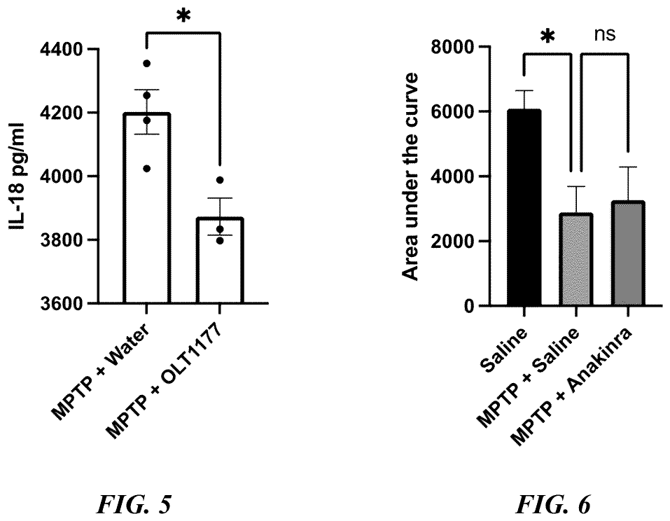

The TNFα RNA fold change is shown in FIG. 4. The results show that dapansutrile reduces the expression of TNFα RNA in the brain.

### Example 4. Dapansutrile Reduces the Levels of IL-18 in the Brain

Mice were treated with dapansutrile or saline and injected with MPTP as described in Example 2.

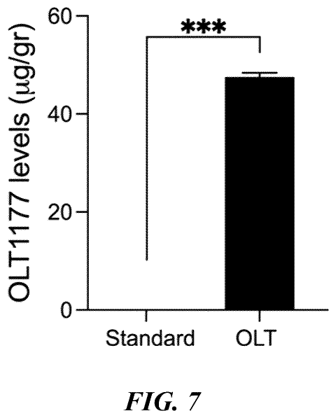

At three days after MPTP, mice were sacrificed and levels of IL-18 in the brain were measured. Briefly, the brain was removed from the mouse and a piece of the striatum was homogenized. Levels of proteins were normalized to 2 mg/ml and IL-18 was measured using the mouse IL-18 ELISA from R and D Systems. We found that treatment with dapansutrile reduced the levels of IL-18 in the brain in comparison with mice treated with water (FIG. 5).

### Example 5. Anakinra does not Produce Functional Improvement

Anakinra is Interleukin-1 receptor antagonist (IL-1Ra). Anakinra (17.3 kDa) differs from native human IL-1Ra (23-25 kDa) by the addition of a methionine at the amino terminus and the absence of glycosylation.

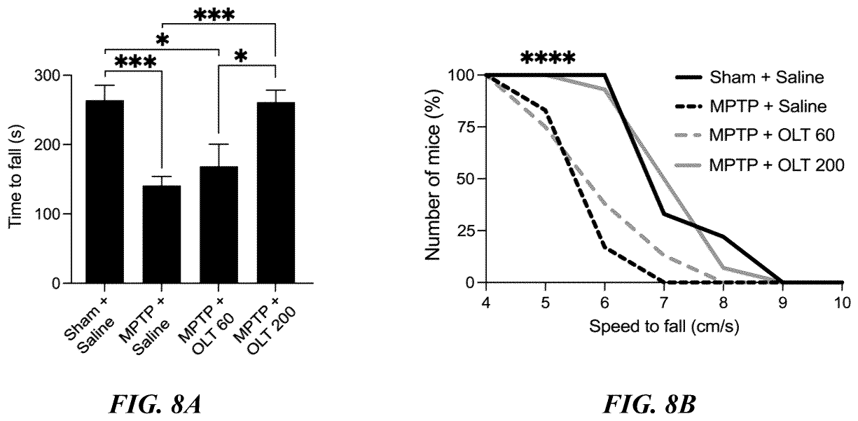

After demonstrating that dapansutrile has a beneficial affect at functional level, we studied the effect of anakinra on rotarod performance. Mice were treated with anakinra at a 20 mg/kg dose 30 minutes before MPTP. We observed that administration of anakinra does not protect the brain from the functional effects of MPTP. Mice treated with anakinra showed the same latency to fall that control mice treated with saline (FIG. 6). We attribute these results to the low penetrance of anakinra into the brain. We do not believe it is worth increasing the dose of anakinra since the CSF penetration of anakinra in non-human primates is only 0.2-0.3% (Fox et al, J. Neuroimmunol. 223: 138-140, 2010).

### Example 6. Dapansutrile Readily Crosses the Blood-Brain Barrier

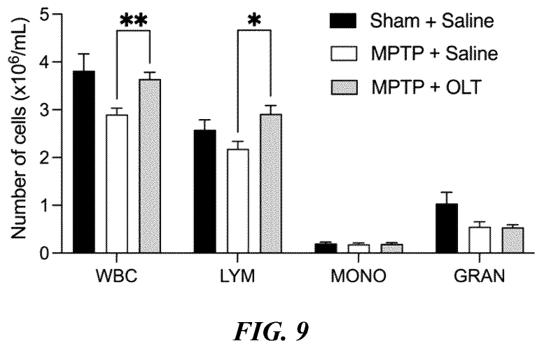

We first demonstrated that dapansutrile crosses the blood-brain barrier (BBB) and achieves therapeutic levels in the brain compartment. Mice were treated with dapansutrile by ad libitum consumption of dapansutrile-fortified feed pellets (7.5 g/kg) for 21 days. Control mice received a matching unfortified diet without dapansutrile. Mice were monitored during the study for any changes in physiological parameters, including irregular weight gain or loss as well as changes in body temperature. On day 21, mice were sacrificed, and brains were isolated. Dapansutrile measurements in the whole brain homogenates were performed at Syneos Health (Princeton, NJ). Five mice per group were assessed. As shown in FIG. 7, dapansutrile reached levels of 47 μg/g in the brain after 21 days of oral administration. Oral dapansutrile readily crosses the BBB and maintains a level in the brain at least 30 times higher than the concentration required to inhibit the NLRP3 assembly. The ability of dapansutrile to cross BBB even when delivered orally provides a potential advance for the treatment of chronic patients like PD.

### Example 7. Dapansutrile Rescues the Locomotor Impairments Associated with MPTP

Next, we tested whether its administration affects the motor performance of the mice after MPTP-acute administration. For this purpose, the locomotor skills of the mice were assessed using the accelerating rotarod test as described in Example 2. Nine mice were used for the sham+saline group. Twelve mice were used for the MPTP+saline group. In the MPTP+dapansutrile group, nine and fourteen mice were used for the 60 mg/kg and 200 mg/kg doses, respectively.

Dapansutrile was administrated i.p. one hour before the first MPTP injection. Two different doses of dapansutrile were used: 60 mg/kg and 200 mg/kg. Solutions were prepared fresh each day. Administration of dapansutrile was repeated once a day until the end of the experiment. Saline was used as the vehicle for i.p. injections. Since acute MPTP regimen can induce fluid overload due to the repeated i.p. injections, the first dapansutrile administration was performed through oral gavage with the same concentration. Water was used as the vehicle for oral gavage.

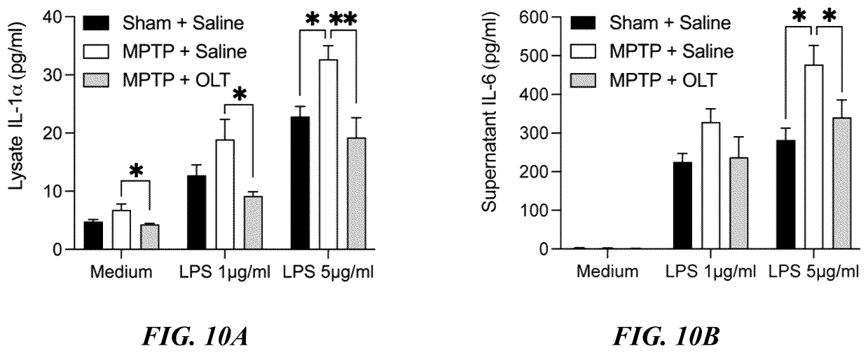

MPTP administration impaired the locomotor performance of the mice after three days, reducing by 120 seconds the time to fall in comparison with sham mice that did not receive MPTP (FIG. 8A). As shown in FIG. 8B, approximately 50% of the mice in the MPTP+saline group failed on the rotating cylinder even before the speed increased to 6 cm/s; none reached 7 cm/s. Mice treated with a lower dose of dapansutrile (60 mg/kg) did not show any significant improvement in comparison with the MPTP+saline group, although a tendency to increase was observed (FIGS. 8A-8B). However, treatment with a higher dose of dapansutrile (200 mg/kg) rescued the locomotor performance associated with MPTP by doubling (p<0.001) the time to fall (FIG. 8A). Moreover, the time to fall on the rotarod device was nearly the same as the sham mice (FIG. 8A). Similar to the sham+saline group, approximately 50% of the mice treated with the 200 mg/kg dose reached the 7 cm/s speed, and 7% of them even reached 8 cm/s (FIG. 8B). Therefore, at the behavioral level, we demonstrate through locomotor performance on the rotarod device that dapansutrile prevented the functional deficits associated to MPTP neurotoxicity.

### Example 8. Dapansutrile Modulates the Systemic Inflammation of MPTP

After demonstrating the protective benefits of dapansutrile on the locomotor activity, we examined the effects of MPTP on the systemic inflammation. Seven days after MPTP-acute administration, peripheral blood and spleen were isolated from each mouse, and hematological changes in the peripheral blood and cytokine production in the spleen were evaluated. Since locomotor function demonstrated that a dose of 200 mg/kg of dapansutrile rescued the effect of MPTP, we administered this dose for the remainder of the experiments. White blood cells, lymphocytes, monocytes, and granulocytes were measured from the peripheral blood using a HemaTrue cell counter (Heska, Loveland, CO). Spleens were isolated and mechanically passed through a 70 μm cell strainer (ThermoFisher) in PBS. Cells were plated in a 96-well plate at a concentration of 200,000 cells/well in RPMI medium (Coring, New York, NY) with 10% FBS. After that, cells were stimulated with LPS at 1 μg/ml and 5 μg/ml. RPMI medium was used as control. After 72 hours, supernatant was isolated to measure the levels of IL-6. Cells were lysed with RIPA buffer (ThermoFisher) supplemented with protease inhibitor (ThermoFisher) to measure IL-1α levels. In both cases, 5-6 mice per group were used.

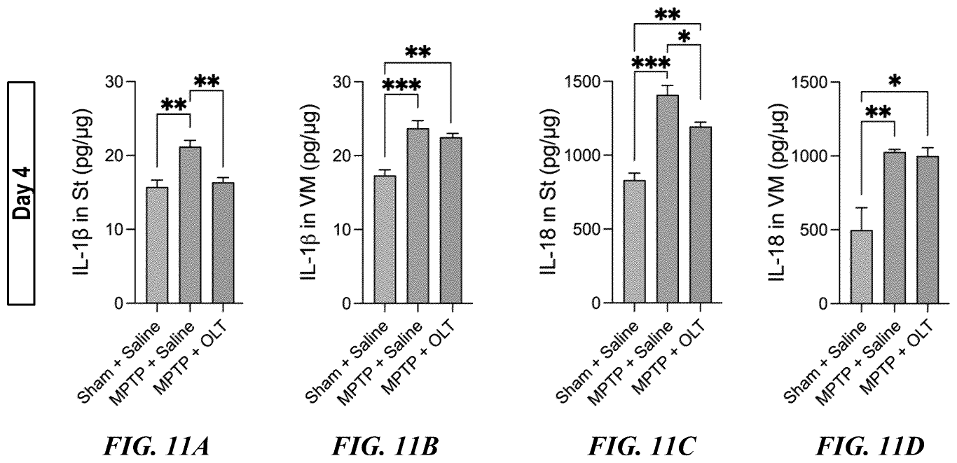

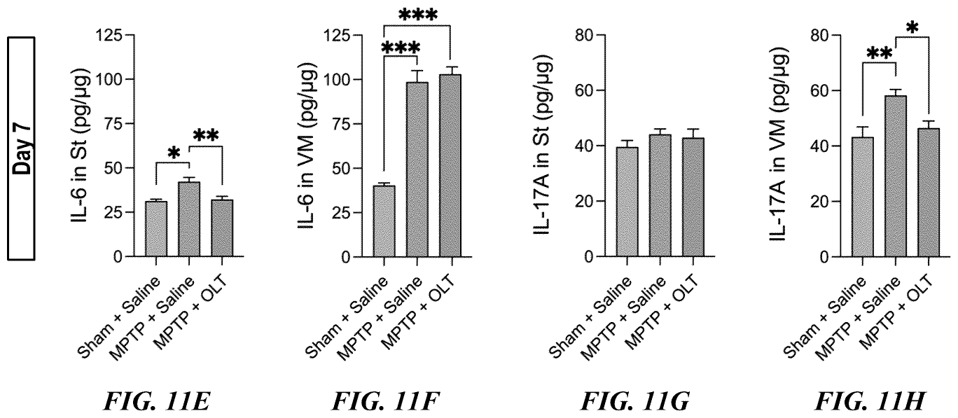

As shown in FIG. 9, MPTP reduced the numbers of circulating white blood cells in the periphery, particularly lymphocytes and granulocytes. However, following the administration of dapansutrile, the number of these cells were significantly increased by 24% and 33%, respectively (FIG. 9). In cultured spleen cells from mice subjected to MPTP, we observed an increased expression of intracellular IL-1α (FIG. 10A). Splenocytes from mice treated with dapansutrile exhibited a significant reduction by 40% of the level of IL-1α. For IL-6, highly similar results were obtained with dapansutrile restoring the effects of MPTP (FIG. 10B).

### Example 9. Administration of Dapansutrile Reduces the Levels of Pro-Inflammatory Cytokines in the Brain

It has been reported that administration of MPTP induces the assembly of NLPR3 inflammasome in the brain. Mice were sacrificed after anesthesia by cervical dislocation at 4 and 7 days after MPTP administration. Brains were collected, and the striatum (St) and ventral midbrain (VM) areas of both hemispheres were excised and frozen in liquid nitrogen. Samples were homogenized in RIPA buffer (Thermo Fisher, Waltham, MA) supplemented with protease inhibitor (Thermo Fisher) using a TissueRuptor (Qiagen, Germantown, MD). Protein concentration was determined using a BCA Protein Assay Kit (Thermo Fisher) according to manufacturer's instructions. Samples were diluted to 2 mg/ml in the same extraction buffer.

Levels of IL-1β, IL-18, IL-6, and IL-17A from brain homogenates were measured by ELISA DuoSet kit (R&D Systems, Minneapolis, MN). ELISA kit for IL-1β detects 92-95% of the 17 kDa form and only 8-5% of the precursor form of this cytokine. 96-well plates (ThermoFisher Scientific) were coated with capture antibody. Samples were incubated for two hours at room temperature. Detection antibodies and streptavidin-HRP were added. TMB ELISA (cBioscience, San Diego, CA) was used as substrate solution. Results were measured in a microplate reader (Bio-Tek, Santa Clara, CA). Final values were normalized by the protein concentration of each sample. 4-5 mice per group were used for each cytokine.

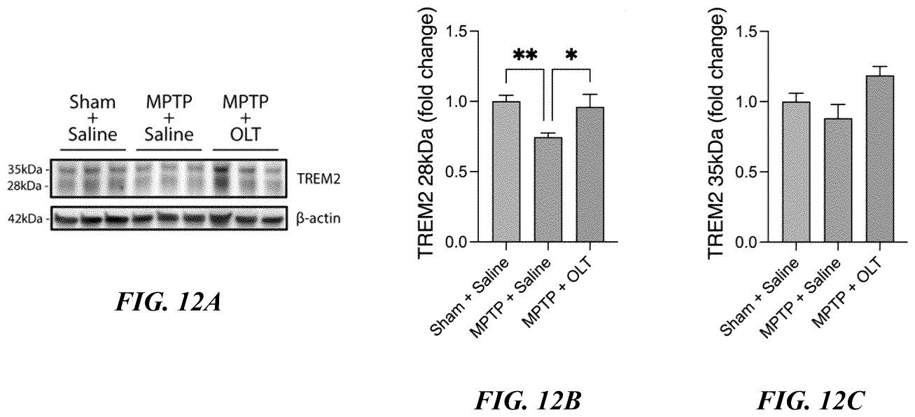

To evaluate the effect of dapansutrile on NLRP3 inhibition, first we measured the levels of IL-1β and IL-18, the downstream cytokines of the NLRP3 inflammasome. IL-1β and IL-18 were measured at the St and VM four days after MPTP-acute administration. Levels of IL-1β significantly increased in the St and VM at day 4, confirming the activation of the NLRP3 inflammasome (FIGS. 11A-11B). In the St, treatment with dapansutrile significantly reduced the levels of IL-1β by 24% at 4 days (FIG. 11A), reaching basal conditions. In the VM, we detected no changes associated to dapansutrile (FIG. 11B). Levels of IL-18 were significantly elevated in both areas of the brain (FIGS. 11C-11D). In the St, mice treated with dapansutrile exhibited a 15% reduction in the levels of IL-18 in comparison with MPTP mice treated with saline (FIG. 11C). The dapansutrile-dependent reduction in IL-18 levels in the St is consistent with results obtained previously in Example 4. As happened with IL-1β, no changes associated to dapansutrile were observed in the VM (FIG. 11D).

After confirming that dapansutrile reduces IL-1β and IL-18, we measured other proinflammatory cytokines that are influenced by IL-1β. We focused on IL-6 and IL-17A, as downstream cytokines of IL-1β, and measured their levels seven days after acute administration of MPTP. For IL-6, we observed that MPTP increased the levels in both areas of the brain (FIGS. 11E-11F). Dapansutrile significantly reduced the levels by 26%, reaching basal conditions in the St (FIG. 11E). No effects were detected at the VM (FIG. 11F). For IL-17A, we did not observe changes due to MPTP in the St (FIG. 11G), however levels significantly increased by 30% (p<0.01) in the VM (FIG. 11H). This supports a previous report showing a similar dynamic of IL-17A in this area of the brain. After treatment, dapansutrile reduced significantly (p<0.05) the levels of IL-17A from 58 pg/μg to 46 pg/μg, nearly the same as in Sham mice (FIG. 11H). Altogether, the data on cytokine dynamics demonstrate that MPTP induces an inflammatory response characteristic of activated NLRP3, and that dapansutrile effectively reduced the levels of these cytokines, modulating the inflammatory response. Changes were observed not only in IL-1β and IL-18 as NLRP3 direct cytokines, but also in IL-6 and IL-17 as downstream cytokines. Thus, in addition to reducing the levels of IL-1β and IL-18 in the brain, dapansutrile also reduced the levels of IL-6 and IL-17A. These data suggest that dapansutrile is effective at modulating the adaptive immune response in PD.

### Example 10. Dapansutrile Preserves the Levels of TREM2 in the Striatum

In this example, we measured the levels of myeloid cell-triggered receptor II (TREM2) after dapansutrile treatment. TREM2 may be a potential target for treating PD. For TREM2, mice were sacrificed at day 4 after MPTP-acute administration. The St was excised, and proteins were extracted and quantified as described in Example 9. 30 μg of protein per sample were resolved in a Mini-PROTEAN TGX 4-20% gradient gel (Bio-Rad, Hercules, CA) in 0.1% SDS running buffer (Bio-Rad). The gel was transferred to a 0.1 μM nitrocellulose membrane (GE Healthcare, Chicago, IL). Membranes were blocked with 5% blocking buffer (Bio-Rad) in TBS 0.1% (v/v) Tween solution for one hour at room temperature. Samples were incubated overnight with primary antibody for mouse TREM2 (1:1000, Cell Signaling, Danvers, MA). Peroxidase-conjugated secondary antibody (Jackson ImmunoResearch, Philadelphia, PA) and chemiluminescence were used to detect the protein concentration. Conjugated antibody against β-actin (Santa Cruz Biotechnology, Dallas, TX) was used to normalize protein concentrations. Protein bands for monomeric and oligomeric forms of each individual mice were quantified using NIH ImageJ software. Four mice per group were used.

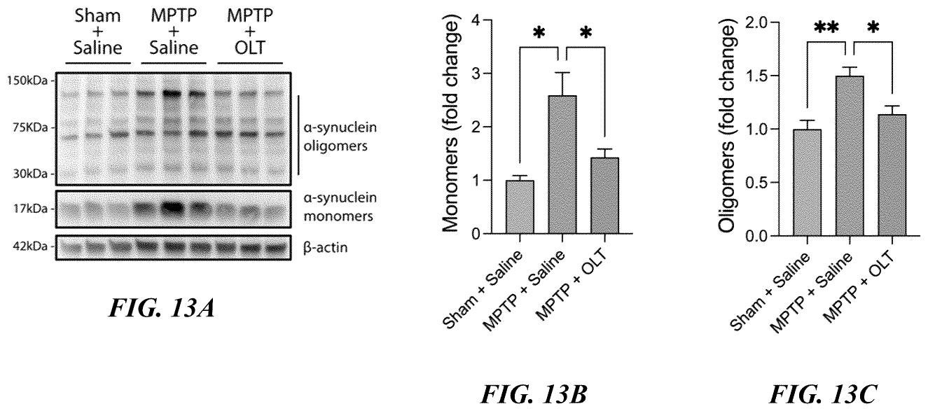

Protein levels of TREM2 were measured in the St area of the brain at day 4 after MPTP-acute administration. MPTP produced a 25% reduction in the levels of nonglycosylated form of TREM2 (28 kDa) (FIGS. 12A-12B). After treatment with dapansutrile, levels of TREM2 were significantly increased, reaching basal condition (FIGS. 12A-12B). In the case of the 35 kDa-glycosylated form of TREM2, no changes associated to MPTP neither dapansutrile were detected (FIGS. 12A, 12C). Although a tendency to increase can be observed after MPTP, differences were not significant. These results showed that NLRP3 inhibition by dapansutrile increased the levels of TREM2 in the St of MPTP mice, correlating with the preservation of dopaminergic neurons. This changes in TREM2 expression did not affect its glycosylation pattern. These findings suppose a new system to modulate TREM2 levels by targeting NLRP3.

### Example 11. Dapansutrile Prevents the Aggregation of α-Synuclein in the Ventral Midbrain

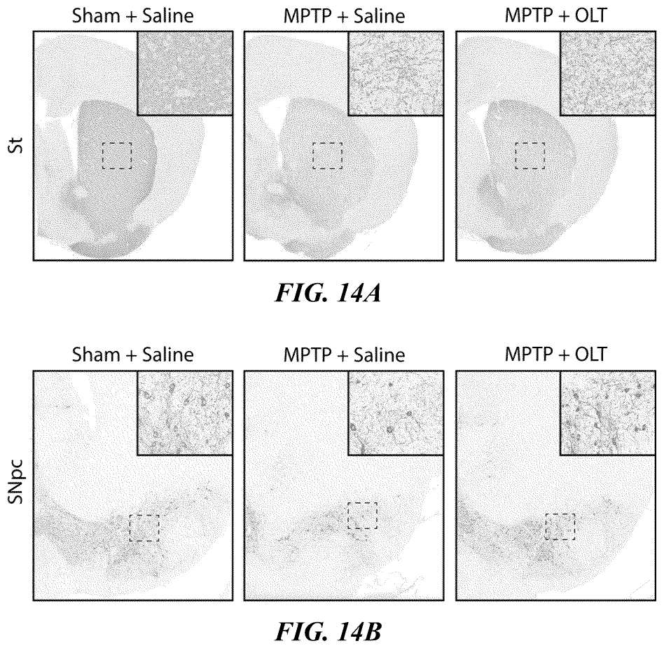

Since MPTP promotes α-synuclein aggregation after subacute regimen, we evaluated the effect of dapansutrile on the levels of this protein. 24 hours after the last injection of MPTP, mice were sacrificed. The VM was excised, and proteins were resolved as described in Example 9. VM was selected as the region to evaluate α-synuclein accumulation according to previous publications. Mouse α-synuclein (1:1000, Cell Signaling) and peroxidase-conjugated secondary antibody (Jackson ImmunoResearch) were used. 4-5 mice per group were used. The levels of α-synuclein were determined by Western blot. As shown in FIGS. 13A-13B, MPTP elevated by 2.5-fold change the levels of the monomeric form of α-synuclein (17 kDa). This correlates with higher aggregation of α-synuclein reflected by the increase (1.5-fold change) of the different oligomeric forms (150-30 kDa) (FIG. 13C). Dapansutrile significantly reduced the levels of both monomers and oligomers of α-synuclein (FIGS. 13B-13C).

### Example 12. Depansutrile Protects Dopaminergic Neurons from MPTP-Induced Cell Death

To evaluate the effect of dapansutrile on dopaminergic neurons, mice were subjected to MPTP acute administration. Seven days after, when the levels of dopaminergic neurons are lower, the nigrostriatal pathway was evaluated by tyrosine hydroxylase (TH) immunostaining to assess the viability of dopaminergic neurons. Mice were perfused with 4% paraformaldehyde (Sigma-Aldrich) in PBS. Brains were removed and immersed in the same perfusion buffer overnight and then cryoprotected with 30% sucrose in 0.1M PBS at 4° C. After dehydration, samples were embedded in paraffin molds for sectioning. Coronal brain sections (4 μm thick) containing the St and SNpc were obtained and transferred to glass microscope slides. After antigen retrieval, slides were incubated with primary anti-TH antibody (1:2000, Abcam, Cambridge, UK) for 30 minutes at room temperature. TH antibody binding was detected using an anti-rabbit HRP-conjugated secondary polymer, followed by chromogenic visualization with diaminobenzidine (DAB, Cell Signaling). Hematoxylin counterstain was used to visualize nuclei. Processing of the samples was performed by Inotiv Boulder (Boulder, CO). Dopaminergic immunoreactivity in the St was calculated by measuring the integrated density by NIH ImageJ software. Stereological counting in the SNpc was performed by manual quantification of TH-positive neurons. Three sections per mice were used. Five mice were used for the sham+saline group, and seven mice were used for the MPTP group.

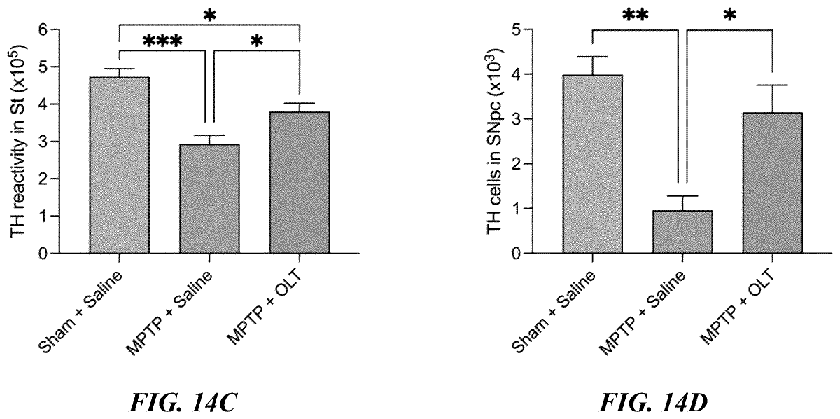

As expected, administration of MPTP reduced the levels of St fibers by 45% in comparison with sham mice (FIGS. 14A, 14C). In SNpc, number of positive neurons were reduced from 4000 to 1000 due to MPTP (FIGS. 14B, 14D). Treatment with dapansutrile protected dopaminergic neurons from detrimental effects of MPTP. In the case of the St, mice treated with dapansutrile showed an increase (p<0.05) in the density of TH-positive fibers in comparison with MPTP mice treated with saline (FIGS. 14A, 14C). TH-positive neurons in the SNpc were also increased 3 times after dapansutrile treatment (FIGS. 14B, 14D). Moreover, for dapansutrile-treated mice, no significant changes against control mice were observed. We conclude that dapansutrile protects dopaminergic neurons by the toxicity caused by MPTP. These findings correlate with the reduction of α-synuclein levels shown in Example 11.

Taken together, these data demonstrate that dapansutrile serves as a treatment to minimize the clinical features of PD through the modulation of the inflammatory response, reduction in α-synuclein levels, and protection of dopaminergic neurons.

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method of treating Parkinson's disease, comprising the step of:
administering to a subject suffering from Parkinson's disease an effective amount of dapansutrile, or a pharmaceutically acceptable solvate thereof.

2. The method according to claim 1, wherein said compound is administered by systemic administration.

3. The method according to claim 1, wherein said compound is administered by oral administration.

4. The method according to claim 1, wherein the method improves one or more movement-related symptoms.

5. The method according to claim 4, wherein said movement-related symptoms are selected from the group consisting of: shaking, rigidity, slowness of movement, difficulty with fine motor skills, walking, and gait.

6. The method according to claim 3, wherein the method improves one or more movement-related symptoms.

7. The method according to claim 6, wherein said movement-related symptoms are selected from the group consisting of: shaking, rigidity, slowness of movement, difficulty with fine motor skills, walking, and gait.
